(19) United States
(12) Patent Application Publication
(10) Pub. No.: US 2024/0404629 A1
(43) Pub. Date: Dec. 5, 2024

(54) DATA PROCESSING METHOD AND APPARATUS FOR VIRUS PROTEIN MUTATION PREDICTION

(71) Applicant: Beijing Youzhuju Network Technology Co., Ltd., Beijing (CN)

(72) Inventors: Zhenqiao SONG, Beijing (CN); Fei YE, Beijing (CN); Xiaoqin TAN, Beijing (CN)

(21) Appl. No.: 18/677,673

(22) Filed: May 29, 2024

(30) Foreign Application Priority Data
May 30, 2023 (CN) 202310632118.5

(51) Int. Cl. G16B 20/20; G06F 30/27; G16B 30/10; G16B 40/20

(52) U.S. Cl. CPC G16B 20/20; G06F 30/27; G16B 30/10; G16B 40/20

(57) ABSTRACT

Embodiments of the present disclosure relate to virus protein mutation site discovery and variant design. There is proposed a data processing method for virus protein mutation prediction, comprising acquiring relevant information about a virus protein sequence for prediction, wherein the relevant information includes amino acid type information, amino acid position information, and corresponding fitness information in the virus protein sequence, and based on the relevant information about the virus protein sequence for prediction, predicting an amino acid mutation site in the virus protein sequence by using a neural network.

### CROSS-REFERENCE TO RELATED APPLICATIONS

This application claims priority to and is based on a Chinese application with an application No. 202310632118.5 and a filing date of May 30, 2023, the aforementioned application is hereby incorporated by reference in its entirety.

### FIELD OF THE INVENTION

The disclosure relates to the field of data processing, especially relates to data processing related to virus protein.

### BACKGROUND

Virus proteins, such as influenza viruses, HIV and SARS-COV-2, have a high degree of variability, resulting in emergence of new variants, which have higher adaptabilities, causing repeated epidemic outbreaks, the adaptability refers to the ability of virus proteins to replicate, propagate and infect. Mutation observed in the Variants of interest (VOIs) or Variants of Concern (VOCs) are associated with increased propagation and reduced effectiveness of antibodies and immune responses. Thus, it is critical for dealing with outbreaks to determine key mutation sites and predict dominant variants quickly and accurately. In addition, understanding the adaptability of virus proteins plays a key role in the rational design of vaccines.

### DISCLOSURE OF THE INVENTION

The DISCLOSURE OF THE INVENTION section is provided to introduce the concepts in a brief form, which will be described in detail in the following specific embodiments. The DISCLOSURE OF THE INVENTION section is not intended to identify key features or essential features of the claimed technical solution, nor to limit the scope of the claimed technical solution.

In a first aspect of the disclosure, there is provided a data processing method for virus protein mutation prediction, comprising: acquiring relevant information about a virus protein sequence for prediction, wherein the relevant information includes amino acid type information, amino acid position information, and corresponding fitness information about the virus protein sequence, and based on the relevant information about the virus protein sequence for prediction, predicting an amino acid mutation site in the virus protein sequence by using a neural network.

In a second aspect of the present disclosure, there is provided a data processing apparatus for virus protein mutation prediction, comprising: an acquisition unit configured to acquire relevant information about a virus protein sequence for prediction, wherein the relevant information includes amino acid type information, amino acid position information, and corresponding fitness information about the virus protein sequence, and a mutation position prediction unit configured to, based on the relevant information about the virus protein sequence for prediction, predict an amino acid mutation site in the virus protein sequence by using a neural network.

In a third aspect of the present disclosure, there is provided an electronic device, which may include: a memory; and a processor coupled with the memory, wherein the processor is configured to, based on instructions stored in the memory, execute the method according to any one of the embodiments of the present disclosure.

In a fourth aspect of the present disclosure, there is provided a computer-readable storage medium having a computer program stored thereon, wherein the program, when executed by a processor, causes the method according to any one of the embodiments of the present disclosure to be implemented.

In a fifth aspect of the present disclosure, there is provided a computer program product including instructions that, when executed by a processor, cause the method according to any one of the embodiments of the present disclosure to be implemented.

In a sixth aspect of the present disclosure, there is provided a computer program including program codes that, when executed by a processor, cause the method according to any one of the embodiments of the present disclosure to be implemented.

Through the following detailed description of exemplary embodiments of the present disclosure with reference to the drawings, other features, aspects, and advantages of the present disclosure will become clear.

### DESCRIPTION OF THE DRAWINGS

Preferred embodiments of the present disclosure are described below with reference to the drawings. The drawings illustrated here are used to provide a further understanding of the present disclosure, and together with the following detailed description, are incorporated in and form a part of the specification, to explain the present disclosure. It should be understood that the drawings in the following description relate to only some embodiments of the present disclosure, and do not constitute a limitation on the present disclosure. In the drawings:

FIG. 1 shows a conceptual illustration of a virus protein mutation prediction according to an embodiment of the present disclosure.

FIG. 2 shows a flowchart of a method for virus protein mutation prediction according to an embodiment of the present disclosure.

FIG. 3 shows a block diagram of an apparatus for a virus protein mutation prediction according to an embodiment of the present disclosure.

FIG. 4A shows an exemplary implementation of a virus protein mutation prediction according to an embodiment of the present disclosure.

FIG. 4B and FIG. 4C show advantageous effects of virus protein mutation prediction in accordance with embodiments of the present disclosure.

FIG. 5 shows a block diagram of some embodiments of the electronic device of the present disclosure.

FIG. 6 shows a block diagram of another some embodiment of the electronic device of the present disclosure.

It should be understood that, for ease of description, the sizes of various parts shown in the drawings are not necessarily drawn to actual scale. The same or similar reference numerals in the drawings are used to denote the same or similar components. Therefore, once an item has been defined in one of the drawings, it may not be further discussed in the subsequent drawings.

### DETAILED DESCRIPTION OF PREFERRED EMBODIMENTS

The technical solutions in the embodiments of the present disclosure are clearly and completely described below with reference to the drawings of the embodiments of the present disclosure. However, apparently, the embodiments described are merely some embodiments of the present disclosure rather than all the embodiments. The following description of the embodiments is actually merely illustrative, and in no way serves as any limitation to the present disclosure and application or use thereof. It should be understood that the present disclosure may be implemented in various forms and should not be construed as being limited to the embodiments set forth here.

It should be understood that the various steps described in the method implementations of the present disclosure may be performed in different orders, and/or performed in parallel. Furthermore, additional steps may be included and/or the execution of the illustrated steps may be omitted in the method implementations. The scope of the present disclosure is not limited in this respect. Unless specifically stated otherwise, the relative arrangement of components and steps, numerical expressions, and numerical values set forth in these embodiments should be construed as merely exemplary, and do not limit the scope of the present disclosure.

The term “include” and the variations thereof used in the present disclosure are open-ended terms that include at least subsequent elements/features but do not exclude other elements/features, that is, “including but not limited to”. In addition, the term “comprise” and the variations thereof used in the present disclosure are open-ended terms that comprise at least subsequent elements/features but do not exclude other elements/features, that is, “comprising but not limited to”. In the context of the present disclosure, “include” has the same meaning as “comprise”. The term “based on” means “at least partially based on”.

The terms “one embodiment”, “some embodiments”, or “an embodiment” described throughout the specification means that the specific features, structures, or characteristics described in connection with the embodiments are included in at least one embodiment of the present invention. For example, the term “one embodiment” means “at least one embodiment”. The term “another embodiment” means “at least one another embodiment”. The term “some embodiments” means “at least some embodiments”. Moreover, the phrases “in one embodiment”, “in some embodiments”, or “in an embodiment” appearing in various places throughout the specification do not necessarily all refer to the same embodiment, but may also refer to the same embodiment. It should be noted that the modifiers “one” and “a plurality of” mentioned in the present disclosure are illustrative and not restrictive, and those skilled in the art should understand that unless the context clearly indicates otherwise, the modifiers should be understood as “one or more”.

It should be noted that concepts such as “first” and “second” mentioned in the present disclosure are only used to distinguish different apparatuses, modules, or units, and are not used to limit the sequence of functions performed by these apparatuses, modules, or units or interdependence. Unless otherwise specified, concepts such as “first” and “second” are not intended to imply that the objects described in this way must be in a given order in terms of time, space, or ranking, or in any other given order.

Protein is the material basis of life, and is the main support of life activity. The three-dimensional structure, functional characteristics and physicochemical properties, etc. of the protein are determined by the amino acid sequence, and the amino acid is a basic unit of the protein. Protein mutations generally refer to amino acid mutations in the amino acid sequence constituting the protein, which may result in variants of the protein.

Currently, many existing methods can be used for evaluating diversity and corresponding variation effects of proteins and their corresponding variants. However, most methods are not specific to virus proteins, and there may exist ambiguity in the prediction due to the lack of virus protein data sets. In addition, the existing method cannot effectively or accurately capture the key mutation site in the protein which is critical to the adaptability. In particular, the machine learning model currently used for rational virus variant generation has the following three limitations: (1) the modelling of the mutation position is not much concerned. The mutation position is a conservative site in the virus protein, affecting the function and folding of the virus protein. Furthermore, the mutation position is very sparse in the virus protein sequence. The average protein length was 500 amino acids, and even for the SARS-COV-2 spike protein length was 1273 amino acids, but less than 10% of the positions were functional for virus suitability. Therefore, definition of modelling key positions can obviously reduce the huge searching space and save time. (2) An epistatic effect is ubiquitous, and the combinatorial mutation is more important than the single-point mutation in the real world. The machine learning methods such as CSCS, EVmutation and DeepSequence are not suitable for the combinatorial mutation. (3) There is few methods to discuss the relationship between the model and high-order dependency of the residues.

In view of this, the present disclosure provides improved virus protein mutation prediction, in particular improved prediction of mutation sites and/or mutation conditions in virus proteins. In the context of the present disclosure, the mutation site may also correspond to a position in the virus protein where a mutation occurs, referred to as a mutation position, both can be used interchangeably throughout the text.

On one hand, considering importance of the mutation position, especially the key mutation site, in the virus protein, the present disclosure proposes an improved prediction of mutation positions in the virus protein, specifically, it can directly find a key position in the virus protein which plays a role in the virus adaptability as the mutation position, which obviously reduces the search space, reduces the processing cost, and enhance the operation efficiency.

On the other hand, the present disclosure further proposes improved prediction of a virus protein mutation condition, especially prediction of amino acid mutation result in the virus protein. Specifically, by using relevant information about the amino acid in the virus protein, especially including but not limited to at least one of amino acid context information and amino acid correlation information in the virus protein, etc., it can identify the amino acid mutation condition, specifically identify the optimal mutation result of the amino acid, therefore, an advantageous amino acid at a specific position with a given adaptive feature can be identified.

In particular, the improved virus protein mutation prediction proposed in the present disclosure can predict an amino acid mutation condition in a virus protein based on the predicted virus protein mutation position by utilizing amino acid context information and amino acid correlation information in the virus protein. Considering that the predicted virus protein mutation positions are significantly less than overall information of the virus protein, the processing overhead for the virus protein mutation prediction based on such positions is also further effectively reduced, and improved amino acid mutation results can also be identified. In this way, it is possible to improve the prediction of variants with higher adaptivity and to depict the adaptive landscape of the variants.

The embodiments of the present disclosure will be described in detail below with reference to the accompanying drawings, but the present disclosure is not limited to these specific embodiments. The following specific embodiments can be combined with each other, and the same or similar concepts or processes may not be described again in some embodiments. Furthermore, in one or more embodiments, specific features, structures or characteristics may be combined in any suitable manner that will be apparent to one of ordinary skilled in the art from this disclosure.

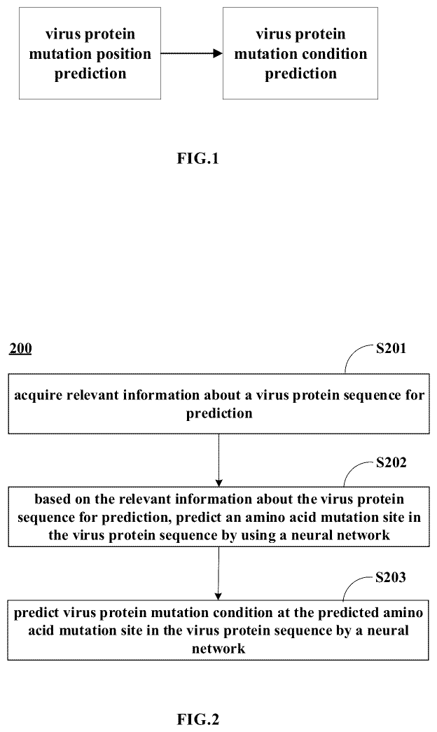

A general conceptual diagram of prediction of virus protein mutation according to an embodiment of the present disclosure will be described below with reference to the accompanying drawings. As shown in FIG. 1, in the virus protein mutation prediction proposed in the present disclosure, two stages may be included: virus protein mutation position prediction, and virus protein mutation condition prediction, wherein the virus protein mutation position prediction may refer to, based on a given virus protein sequence, predicting a position where an amino acid mutation may occur. By way of example, the virus protein sequence may comprise a specific number of various types of amino acids, and the virus protein mutation position prediction may refer to prediction of a position of the amino acid in the virus protein sequence which would mutate. The virus protein mutation condition prediction may refer to, based on a given virus protein sequence, predicting the mutation condition of an amino acid, including, for example, predicting what type of amino acid can an amino acid at a specific position be transformed to.

In some embodiments, mutation position prediction and virus protein mutation condition prediction may be performed in a variety of appropriate ways, such as by appropriate data processing algorithms, models, and the like. By way of example, embodiments of the present disclosure may be implemented by a variety of suitable machine learning methods, models, etc., such as deep learning models, neural network models, etc. In particular, in some embodiments, at least one of mutation position prediction and virus protein mutation condition prediction can be performed iteratively, and the iteration terminates after a particular termination condition is satisfied, and the resulting result is the prediction result. By way of example, the iteration termination condition may be that a particular number of iterations is reached, an iteration loss is below a particular threshold, an iteration loss for a particular number of consecutive iterations is below a particular threshold or within a particular range, or any other appropriate termination condition, which will not be described in more detail herein.

In some embodiments, both mutation position prediction and virus protein mutation condition prediction may also be accomplished by an overall model or network. For example, in a neural network model implementation, an overall loss function is constructed, the input of the neural network model may include at least a virus protein sequence containing a particular number of amino acids, and the output may be a mutation prediction result of the virus protein sequence. By way of example, it is possible to predict information related to the mutation condition in the virus protein sequence, such as a position where the mutation will occur, the amino acid mutation type, the amino acid mutation probability, etc., and it may predict the mutated virus protein sequence, such as the identified variant.

In embodiments of the present disclosure, a neural network model for virus protein mutation prediction can be trained in an appropriate manner. In particular, the neural network training can be efficiently performed using virus protein sequence input and virus protein sequence mutation output as training data, thereby optimizing parameters of the neural network model for the virus protein sequence. Such trained neural network model can be effectively used for efficient and accurate mutation prediction of virus protein sequence.

The specific implementation of the virus protein mutation prediction according to embodiments of the present disclosure will be further described below.

FIG. 2 shows a flowchart of a method for virus protein mutation prediction according to an embodiment of the present disclosure. In the method 200, at step S201, acquiring relevant information about a virus protein sequence for prediction, and at step S202, based on the relevant information about the virus protein sequence for prediction, predicting a mutation position in the virus protein sequence by using a neural network.

In some embodiments, the relevant information about the virus protein sequence for prediction may include amino acid type information, amino acid position information about the virus protein sequence, wherein the amino acid type information may indicate the type of each amino acid contained in the virus protein sequence, and the amino acid position information indicates the position of each amino acid contained in the virus protein sequence.

Additionally, the relevant information about the virus protein sequence for prediction may also include fitness information corresponding to the virus protein sequence, which may be represented by a fitness tag sequence corresponding to the virus protein sequence, wherein each amino acid in the virus protein sequence has a corresponding fitness tag. The fitness information can be generated by performing artificial mutation on the virus protein, which indicates the fitness change trend caused by mutation, wherein the fitness tag being a first specific value (e.g., 1) indicates that the mutation causes the fitness of the protein to become better, and the fitness tag being a second specific value (e.g., 0) indicates that the mutation causes the fitness of the protein to become poor.

In this case, in some embodiments of the present disclosure, the acquired input may include an amino acid sequence of a virus protein, which may be referred to as a wild virus protein sequence, and a corresponding mutation fitness tag sequence, wherein each tag in the mutation fitness tag sequence indicates the mutation tag of the corresponding amino acid in the virus protein sequence, for example, the mutation sequence fitness tag being 1 indicates a beneficial mutation that the mutation fitness of the corresponding amino acid is better than that of the wild sequence, the mutation sequence fitness tag being 0 indicates a deleterious mutation that the mutation fitness of the corresponding amino acid is lower than that of the wild sequence. Of course, the fitness tags for the mutation sequence can also be other appropriate values, as long as different mutation conditions can be distinguished. In some embodiments, the virus protein sequence as an input may comprise only a sequence of amino acids having specific fitness tag values, and in particular, may comprise a sequence of amino acids corresponding to fitness tag values (e.g., fitness tag values of 1) indicating that the mutation fitness becomes better.

According to embodiments of the present disclosure, each of the amino acid type information, amino acid position information, and fitness information about the virus protein sequence can be represented in any suitable manner, for example, represented in the manner of Embedding label. The Embedding label manner may be known in the art, for example, a distributed representation method, i.e., distributedly representing the original input data by a linear combination of a series of features, so that the desired data can be more accurately represented. The embedded label may be implemented in various ways and will not be described in detail herein.

According to embodiments of the present disclosure, the relevant information about the virus protein sequence for prediction can be encoded, and in particular, the virus protein sequence including the virus amino acid type information, the position information, and the corresponding fitness sequence can be encoded. The encoding may be performed using various appropriate encoding methods, and may be encoded as feature vectors of different dimensions. Depending on the encoding mode, the dimensions of the feature vectors before and after encoding can be different. For example, the dimension may be the same as or different from that of the wild sequence. By way of example, an encoder may be implemented in a variety of appropriate forms, for example, can be composed of a particular number of transformer encoder layers, each layer is composed of a self-attention block and a location-aware feed-forward block, which can be various suitable structures and can be implemented by various techniques in the art, and will not be described in detail herein.

According to embodiments of the present disclosure, a mutation position in the virus protein sequence can be predicted based on the relevant information about the input virus protein sequence or the relevant information about the encoded input virus protein sequence. The prediction may be implemented using an appropriate model, algorithm, function, or the like, and in some embodiments, the prediction may be based on a neural network using an appropriate loss function. In particular, prediction can be performed by using a SoftMax function, as an example, the prediction can be performed by using a loss function for a prediction model constructed by a particular SoftMax function, based on the relevant information about the input virus protein sequence or the relevant information about the encoded virus protein sequence, and parameters of the prediction model. In some other embodiments, the SoftMax function may be implemented as a SoftMax layer in the neural network, so that the relevant information about the input virus protein sequence or the relevant information about the encoded virus protein sequence may be input into the SoftMax layer to obtain a prediction result.

According to embodiments of the present disclosure, depending on the implementation of the SoftMax function, the prediction result of a virus protein mutation position may be in various forms. In some embodiments, the obtained prediction result may be a binarized representation that directly indicates whether the amino acid at each amino acid position in the virus protein sequence is variable, e.g., 1 indicates variable, and 0 indicates invariable. As an example, the obtained prediction result may be a vector corresponding to the virus protein sequence, in which each element is binarized to indicate whether the amino acid at the corresponding amino acid position is variable, thereby indicating the amino acid mutation position.

In some other embodiments, the probability of amino acid mutation occurring at each position in the virus protein sequence can be obtained as a result of the amino acid mutation position prediction. The resulting prediction result sequence may correspond to the input virus protein sequence, each position corresponding to the probability of amino acid mutation at that position, thereby determining positions in the virus protein sequence at which mutation may occur. For example, in a case where the probability is greater than a specific probability threshold (first probability threshold), the protein mutation probability at the position is considered large and the position may be labelled as a mutation position; in a case where the probability is less than a specific probability threshold (the first probability threshold), the protein mutation probability can be considered small and the position may not be labelled as a mutation position. According to embodiments of the present disclosure, in particular, the predicted probability can also be further processed to simplify the obtained result while still highlighting the predicted mutation position. In some embodiments, the mutation positions in the virus protein sequence can be obtained based on the predicted probabilities of mutation sites in the virus protein sequence by using a mask. For example, by a binarization process, for example, comparing the predicted mutation probability at each position with a particular threshold, and setting a mutation probability greater than or equal to the particular threshold to 1, and setting a mutation probability less than the particular threshold value to 0, it is possible to obtain a more simplified sequence highlighting the predicted mutation positions. It should be noted that the binarized values may also be other appropriate values, as long as different probabilities can be distinguished, which will not be described in detail herein.

Thereby, through the position prediction according to embodiments of the invention, a key position in the virus protein which plays a role in the virus adaptability, especially the amino acid mutation position, can be directly found, so that the searching space can be obviously reduced, and the searching speed can be obviously enhanced, and the ability of the model for detecting the mutation site can be improved.

According to embodiments of the present disclosure, it can further predict the virus protein mutation condition based on the predicted amino acid mutation positions in the virus protein sequence, such as step S203. Here, the virus protein mutation condition may refer to mutation conditions of amino acids in the virus protein sequence, for example, at the predicted mutation positions in the protein, what type of amino acid can the amino acid mutate to. The prediction may be implemented using appropriate models, algorithms, functions, etc., such as deep learning, such as neural networks, etc. In some embodiments, prediction can be made based on a neural network using an appropriate loss function. In particular, the amino acid mutation condition at the amino acid mutation site in the virus protein sequence can be predicted by using a SoftMax function, for example, by using a mutation prediction loss function based on a SoftMax function.

Depending on the applied prediction mode, the predicted mutation condition may be indicated by various appropriate forms of information. In some embodiments, the amino acid mutation condition prediction may involve predicting the probability distribution of the amino acid mutation condition, i.e., the probability of mutating to what type of amino acid, so that the amino acid mutation probability distribution can be predicted as the prediction result. In some other examples, a specific amino acid mutation may further be selected from the predicted amino acid mutation probability distribution as the prediction result, for example, an amino acid mutation with the highest probability, or an amino acid mutation with a probability higher than a particular threshold (second threshold), or the like, can be selected as the prediction result. Thus, it is possible to recognize an amino acid at the mutation site in the virus protein sequence that is advantageous for a given adaptivity feature.

According to embodiments of the present disclosure, prediction of the amino acid mutation condition can be implemented based on at least one of amino acid context information and amino acid correlation information in a virus protein sequence, preferably using both amino acid context information and amino acid correlation information. The prediction can be implemented in various appropriate ways, in particular, the prediction can be implemented by using a mutation prediction loss function that can be constructed based on at least one of amino acid context information and amino acid correlation information in the virus protein sequence. In some embodiments, the mutation prediction loss function may be constructed by at least one of a first loss function for prediction based on amino acid context information and a second loss function for prediction based on amino acid correlation information. In particular, in some embodiments, the mutation prediction loss function can be constructed by weighted combination of a first loss function for prediction based on amino acid context information and a second loss function for prediction based on amino acid correlation information, so that the mutation condition is thereby predicted based on the constructed loss function. By way of example, the contributions of the first loss function and the second loss function can be set with specific weights, thereby optimizing the mutation prediction loss function.

According to embodiments of the present disclosure, the amino acid context information in the virus protein sequence may include information related to individual amino acids in the virus protein sequence. In some embodiments, the amino acid context information includes amino acid position information and amino acid-relevant information in the virus protein sequence to be predicted, specifically, amino acid position information may be, in particular, amino acid mutation position information in the virus protein sequence, which may be determined as previously described, and will not be described in detail here, and for mutation condition prediction, relevant information indicating or reflecting the probability distribution of mutation or evolution of the amino acid itself in the virus protein sequence can be obtained based on the amino acid context information, and may also be referred to as relevant information about the amino acid self-mutation/evolution in the virus protein sequence.

According to embodiments of the present disclosure, prediction based on the amino acid context information can be calculated by various appropriate algorithms, functions, etc. In some embodiments, the calculation may be implemented based on a neural network by using an appropriate loss function. By way of example, a SoftMax function can be used for the calculation, in particular, by using a loss function (i.e., a first loss function) for a prediction model constructed based on the SoftMax function, the probability distribution can be acquired based on the amino acid position information, in particular the mutation position information as predicted above, and the model parameters. In some embodiments, for each amino acid in the virus protein sequence, especially the amino acid at the mutation position predicted as above, the position distribution information can be acquired based on the position information of the amino acid by using a SoftMax function, and the amino acid mutation probability distribution in the virus protein sequence can be acquired based on combination of the acquired position distribution information and amino acid type information (e.g., Embedding label), e.g., by using a SoftMax function.

According to embodiments of the present disclosure, the amino acid correlation information in the virus protein sequence may include relevant information about coevolution of amino acids, which may be indicative of mutual correlation or coupling between a specific amino acid and other amino acids in the virus protein sequence, and from which a possible probability distribution can be obtained, for example, indicative of coupling or co-evolution of both. The amino acid correlation information in the virus protein sequence may include various appropriate information, particularly residue dependency in the virus protein sequence. By way of example, the residue dependency may refer to global pairwise dependency between residues in multiple sequence alignment (MSA).

In some of embodiments of the present disclosure, the residue dependency can be acquired by using a model for predicting inter-residue distance (e.g., CCMPred), and additionally can be acquired by using a Markov Random Field (MRF) method, for example, by using a model specified by the Markov random field method. As an example, the energy of the virus protein sequence can be modelled, for example, as an energy function, which can serve as a sum of all pairwise coupling constraints and single position constraints, whereby the residue dependent relationship in the virus protein sequence can be appropriately determined. For example, the residue dependent relationship may be derived from pairwise coupling constraint terms in the modelled energy function, and in particular, when the MRF model is suitable for data with appropriate regularization, the residue dependent relationships in the protein sequence may be interpreted by direct coupling terms.

According to embodiments of the present disclosure, the prediction based on amino acid correlation information, such as the residue dependent relationship, can be implemented in a variety of appropriate ways, such as using appropriate algorithms, models, and the like. In some embodiments, prediction can be made based on a neural network by using an appropriate loss function. As an example, a softmax function may be used. In some examples, by using a loss function (i.e., a second loss function) for a prediction model constructed based on the SoftMax function, the probability distribution of pairwise coupling can be acquired at least based on the amino acid residue dependent relationship and the model parameters. In some other examples, the probability distribution of pairwise coupling may also be obtained based on single position constraints for amino acids, the residue dependent relationship, and model parameters. By way of example, the evolution coupling can be identified by using Markov random field (MRF) and by learning multiple sequence comparisons among homologous virus protein sequences, so that a coupling matrix and site preference vectors can be obtained, wherein the former can correspond to the dependent relationship and the latter can correspond to self-context information of amino acids.

According to embodiments of the present disclosure, amino acid context information and amino acid correlation information in a virus protein sequence may be acquired based on a homologous virus protein sequence, wherein the homologous virus protein sequence may be generated based on a virus protein sequence for predicting mutation sites in the virus protein sequence. In particular, in some embodiments, the homologous virus protein sequence may have the same or similar amino acid sequence as the virus protein sequence used to identify the protein mutation positions. It can be derived from an existing database containing a large number of virus protein sequences, or evolved from an existing virus protein sequence. In some embodiments, the homologous virus protein sequence may be pre-formatted, for example, may be formatted into a multi-sequence alignment (MSA) format, and then data processing is performed based on the formatted sequence to obtain contextual information of the virus protein sequence and global pairwise dependencies between the residues in the sequence alignment.

Thereby, at least using the amino acid correlation information in the virus protein sequence, even using both the context information in the virus protein sequence and the amino acid correlation information in the virus protein sequence, to perform virus protein mutation prediction, so as to optimize the prediction of amino acid mutation, the amino acid variants in the virus protein sequence can be better produced, thereby producing an improved virus protein sequence. In particular, amino acid type prediction for mutation positions can be performed using context information from virus protein sequences and evolution information from multi-sequence alignment, and one weight parameter is used for controlling a probability distribution loss function predicted based on the context information of the virus protein sequence and a probability distribution loss function predicted based on the correlation information to jointly train the model, so that the most possible probability distribution can be obtained.

According to embodiments of the present disclosure, the amino acid mutation prediction result for a virus protein sequence may be in various appropriate forms. In one embodiment, the result may be the amino acid mutation position, mutation condition probability, etc. of the virus protein sequence as predicted in the above manner, whereby the amino acid mutation of the virus protein sequence can be appropriately determined. For example, the amino acid mutation result may be determined based on the probability of amino acid mutation condition, for example, determined based on the amino acid mutation corresponding to the probability of amino acid mutation condition above a particular threshold. By way of example, for amino acid mutation positions in the virus protein sequence, a respective probability threshold may be set for each amino acid mutation position, or a common probability threshold may be set for all amino acid mutation positions. In another embodiment, an amino acid mutation having the highest amino acid mutation condition probability can also be used as the amino acid mutation result. For example, for amino acid mutation positions in the virus protein sequence, an amino acid mutation having the highest amino acid mutation condition probability can be selected so as to obtain the amino acid mutation result. Therefore, the predicted virus protein variant can be obtained.

The data processing in accordance with embodiments of the present disclosure, particularly data processing related to mutation prediction for virus protein sequences, can be performed in various appropriate ways. In some examples, centralized processing may be performed, for example, by a single processing device or apparatus, such as a variety of appropriate types of servers, processors, graphics processing units (CPUs), or the like. In some other examples, distributed processing may be performed, for example, on a plurality of computing nodes, at least one of which may include a variety of appropriate types of servers, processors, graphics processing units (CPUs), etc., and a part of the data processing is performed on each of the computing nodes, respectively.

According to embodiments of the present disclosure, the foregoing embodiments may be combined in a suitable manner. In particular, a unified or global neural network can be used to directly determine or predict a virus protein sequence, which may mutate, from virus protein sequences. By way of example, the global neural network or model can implement both position prediction and mutation condition prediction, so that the relevant information about the virus protein sequence variation can be obtained from the input relevant information about the virus protein sequence. In some embodiments, a global loss function may be constructed for a global neural network or model, such as a loss function based on a SoftMax function, which may be a weighted combination of a mutation position loss function and an amino acid mutation condition loss function, and the prediction can be performed based on the constructed global loss function, thereby acquiring a mutation prediction result of the virus protein sequence, such as mutation probability distribution, or further processing, etc., as described above, which will not be described in further detail herein.

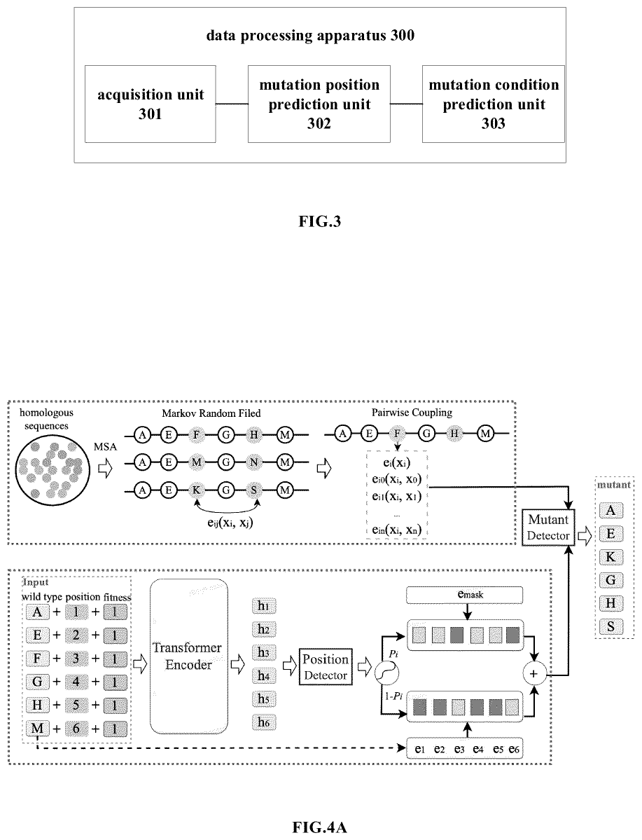

FIG. 3 is a block diagram of a data processing apparatus according to an embodiment of the present disclosure. The data processing apparatus may also be referred to as a data processing apparatus for mutation prediction of a virus protein sequence. The apparatus 300 may include an acquisition unit 301 configured to acquire relevant information about a virus protein sequence for prediction, and a mutation position prediction unit 302 configured to, based on the relevant information about the virus protein sequence for prediction, predict a mutation position in the virus protein sequence by using a neural network.

According to some embodiments of the present disclosure, the mutation position prediction unit 302 may be further configured to.

According to some embodiments of the present disclosure, additionally or alternatively, the mutation position prediction unit 302 may be further configured to predict the amino acid mutation site in the virus protein sequence using a mutation position prediction loss function based on a SoftMax function.

According to some embodiments of the present disclosure, the data processing apparatus 300 may also include a mutation condition prediction unit 303 configured to predict a virus protein mutation condition based on the predict amino acid mutation position in the virus protein sequence.

According to some embodiments of the present disclosure, additionally or alternatively, the mutation condition prediction unit 303 may be further configured to, based on at least one of amino acid context information in the virus protein sequence and amino acid correlation information in the virus protein sequence, predict the amino acid mutation condition in the virus protein sequence at the predicted amino acid mutation site in the virus protein sequence by using a neural network.

According to some embodiments of the present disclosure, additionally or alternatively, the mutation condition prediction unit 303 may be further configured to predict the amino acid mutation condition in the virus protein sequence at the predicted amino acid mutation site in the virus protein sequence by using a neural network, by using a mutation prediction loss function based on a SoftMax function. In some embodiments, the amino acid mutation condition can be predicted by using a neural network. In some embodiments, the amino acid mutation condition may include a variety of appropriate information, and preferably, the amino acid mutation condition involves a probability distribution of an amino acid mutating to a particular amino acid type.

According to some embodiments of the present disclosure, the mutation prediction loss function can be determined based on at least one of a first loss function for prediction based on amino acid context information and a second loss function for prediction based on amino acid correlation information, and preferably, in some embodiments, the mutation prediction loss function may be determined by a weighted combination of the first loss function for prediction based on amino acid context information and the second loss function for prediction based on amino acid correlation information.

According to some embodiments of the present disclosure, the amino acid context information may include position information and amino acid relevant information of the predicted amino acid mutation site in the virus protein sequence, and the first loss function may be constructed based on the amino acid context information, a particular SoftMax function. According to some embodiments of the present disclosure, the amino acid correlation information may include residue dependency in the virus protein sequence obtained based on multiple sequence alignment for the virus protein sequences, and a second loss function may be constructed based on the residue dependency, a particular SoftMax function.

According to some embodiments of the present disclosure, residue dependency in the virus protein sequence can be obtained by constructing an energy function for the virus protein sequence which is constructed as a sum of all pairwise coupling constraints and single position constraints, and determining the residue dependency in the virus protein sequence based on at least the pairwise coupling constraint terms in the energy function.

In some embodiments of the present disclosure, the operations related to the construction of the mutation prediction loss function, such as at least one of the construction of the first loss function, the construction of the second loss function, the residue dependency in the virus protein sequence, and the like may be performed by the mutation condition prediction unit 303, and, of course, may also be performed outside the mutation condition prediction unit 303, for example by other units in the device, even other devices outside of the apparatus, and the operation results can be directly provided to the mutation condition prediction unit 303 for the mutation condition prediction.

It should be noted that the operations or processes performed by the above apparatus and the various units contained therein may be performed as described above, for example as in the corresponding steps described above, and will not be described in more detail herein.

It should be noted that the above-mentioned various units are merely logical modules divided according to specific functions implemented by the units, and are not used to limit specific implementations, for example, the units may be implemented by software, hardware, or a combination of software and hardware. In actual implementation, the above-mentioned various units may be implemented as separate physical entities, or may be implemented by a single entity (for example, a processor (a CPU, a DSP, etc.), or an integrated circuit). In addition, the above-mentioned various units are shown with dotted lines in the drawings to indicate that these units may not actually exist, and the operations/functions implemented by them may be implemented by a processing circuit. In particular, depending on the implementation of operations by the embodiments of the present disclosure, such units can be implemented in a centralized manner or a distributed manner.

In addition, although not shown, the apparatus may further include a memory, which may store various information generated during the operations by the apparatus and the various units included in the apparatus, program and data used for the operations, data to be sent by a communication unit, etc. The memory may be a volatile memory and/or a non-volatile memory. For example, the memory may include, but is not limited to, a random-access memory (RAM), a dynamic random-access memory (DRAM), a static random-access memory (SRAM), a read-only memory (ROM), and a flash memory. Certainly, the memory may alternatively be located outside of the apparatus. Optionally, although not shown, the apparatus may further include a communication unit, which may be used to communicate with other apparatuses. In an example, the communication unit may be implemented in an appropriate manner known in the art, for example, including communication components such as an antenna array and/or a radio frequency link, various types of interfaces, communication units, and the like, which will not be described in detail here. In addition, the apparatus may further include other components not shown, such as a radio frequency link, a baseband processing unit, a network interface, a processor, and a controller, etc., which will not be described in detail here.

It should be noted that, although in the description of the present disclosure, the solution of the present disclosure is mainly described by taking the virus protein sequence as an example, it should be noted that the embodiments of the present disclosure can also be applied to other appropriate types of protein sequences, and similar advantageous technical effects can be achieved.

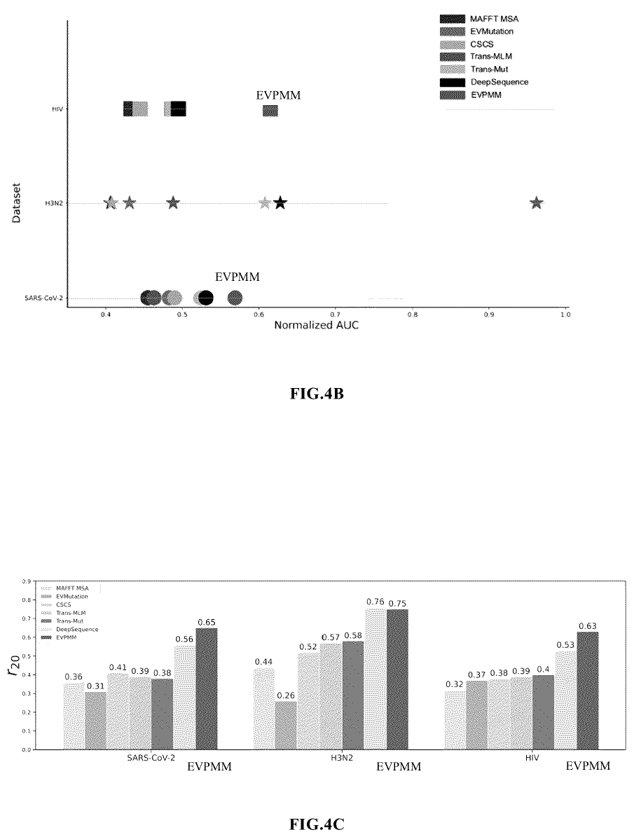

Exemplary implementation for amino acid mutation prediction for a virus protein sequence in accordance with an embodiment of the present disclosure will be described below with reference to FIG. 4A. In this exemplary implementation, the disclosed scheme may be generally a system for amino acid mutation prediction for the virus protein sequence, which may include a mutation position predictor and a mutation condition predictor. The system can be implemented by a neural network model, so that model training can be performed by constructing a suitable loss function, and then more optimized prediction results can be obtained. Potential virus variants with higher adaptive advantages can be found.

Specifically, for the system according to the present disclosure, the input may be a given wild virus strain sequence X=(x1, x2, . . . , xn) and a corresponding fitness tag sequence Z, and the output corresponds to the generated or predicted protein variants Y=(y1, y2, . . . , yn), wherein n is the number of amino acids in the virus sequence. In a case that the system is implemented by a neural network model, the overall training target can be set as follows:

$$L = Lpos + λ Lmut \qquad ( 1 )$$

wherein Lpos may indicate a loss function used in the mutation position prediction, for example, corresponding to the loss function described previously, and Lmut may indicate a loss function in the mutation condition prediction, for example, corresponding to the mutation condition loss function described previously. and λ may be a parameter, which can also be regarded as a weight parameter, and a hyperparameter, which can be appropriately set, for example according to experience. For example, it can be obtained by overall system training, for example, specific training data can be used to train the overall model to determine various appropriate model parameters, especially neural network parameters.

In an exemplary implementation, the system or model, in particular the position prediction stage therein, may be composed of L Transformer encoder layers, each layer can be composed of a self-attention block and a position-aware feed-forward block, which may be defined as:

$$HL = F ( H θ ; θ ) \qquad ( 2 )$$

wherein H0 indicates a virus protein sequence input as the model input, H0={H01, H02, . . . , H0n}, θ indicates parameters used in the encoding process, for example, the parameter used in the encoding process corresponds to the model or various proper algorithms. HL is output of L-th layer of the model, and may be expressed as a code output, may be expressed as HL={HL1, HL2, . . . , HLn}.

In an exemplary implementation, the model input may be in a variety of appropriate forms, such as an Embedding form. As an example, the element at the specific position i in the model input H0 may be as follows:

$$H 0 i = Emb ( xi ) + Emb ( posi ) + Emb ( Z ) \qquad ( 3 )$$

wherein the Emb (xi), Emb (posi) and Emb (Z) respectively indicate virus amino acid type information, virus amino acid position information, and corresponding fitness information about the amino acid at position i in the virus protein sequence, and can be obtained by embedding.

Then, the mutation position can be predicted by the following formula:

$$P \hat{} pos = softmax ( WposHL ) \qquad ( 4 )$$

The above formula can be a function representation of an operation of performing mutation position prediction by a model, a neural network, or the like. Here, Wpos indicates trainable model parameters, especially parameters of a partial model for mutation position prediction in the system model, even partial parameters corresponding to the mutation position prediction in the system model parameters. As an example, P^{\hat{ }}pos can be a two-dimensional one-hot vector indicating whether the amino acid at each position changes, or a mutation probability. Here, the SoftMax function may be a variety of appropriate functions known in the art, and will not be described in detail herein.

A set of amino acids in the virus protein sequence that may change can be obtained, for example, as a subset PS representing possible functional positions, by the mutation position prediction described above.

Then, possible mutated amino acids, such as types of the mutated amino acids, can be predicted based on the set of amino acid mutation positions obtained by the mutation position prediction. By way of example, the probability distribution of a mutated amino acid can be predicted. The probability distribution of the mutated amino acid is composed of two parts: a virus protein sequence and a multi-sequence alignment, these parts can be controlled by a hyperparameter α.

The mutation prediction loss function can be expressed as follows:

$${L}_{mut} = {\sum }_{j ∈ ℙ𝕊} - {P}_{j}^{mut} log {\hat{P}}_{j}^{mut}$$

$${\hat{P}}^{mut} = α * {P}_{mut}^{seq} + ( 1 - α ) * {P}_{mut}^{msa}$$

wherein PS refers to a set of virus protein sequences, in particular a set of positions predicted to be likely to mutate, in particular a set of mutation positions exceeding a particular threshold, and j indicates the position contained in the set. Wherein, Pⱼ^{mut} may be in a variety of suitable forms, for example, in the form of a 20-dimensional one-hot embedded vector. Pₘᵤₜ^{seq} may indicate mutation information predicted based on amino acid self-information in the virus protein sequence. Pₘᵤₜ^{msa} may indicate co-mutation information predicted based on amino acid correlation information in the virus protein sequence. α may be a weight, which may be set to an appropriate value, for example in the range of 0 to 1. In this way, the probability of amino acid mutation can be more accurately determined by considering the self-information about amino acids in the virus protein sequence, and the collaborative information, or the correlation information, or the mutual relationship about amino acids in the virus protein sequence.

In one aspect, the mutation probability distribution can be calculated from the virus protein sequence itself, e.g., using Gumbel SoftMax, as follows:

$${\hat{P}}_{j}^{pos} = gumbel - softmax ( {\hat{P}}_{j}^{pos} )$$

$${\tilde{H}}_{j} = {\tilde{p}}_{j}^{0} * Emb ( {x}_{j} ) + {\tilde{p}}_{j}^{1} * Emb ( [ mask ] )$$

$${P}_{mut}^{seq} = softmax ( {W}^{seq} \tilde{H} )$$

Wherein, \tilde{P}ⱼ⁰ and \tilde{P}ⱼ¹ represent 0 and 1 dimensional values, respectively. Emb ([mask]) represents the embedding of the [mask] symbol. W^{seq} indicates a trainable parameter. The above formula may be a function representation of the mutated amino acid probability distribution predicted from the virus protein sequence itself, which may correspond to the loss function in the prediction.

On the other hand, the mutation probability distribution can be obtained by the multiple sequence alignment. In order to better capture higher-order amino acid covariation, CCMpred based on Markov random field (MRF) specification can be used to model residue dependencies in a virus protein sequence, which are indicative of correlation characteristics among amino acids in the virus protein sequence. The energy function E(X) of the sequence X can defined as the sum of all pairwise coupling constraints eij and single position constraints ei, where i and j are position indices along the virus protein sequence, which may be implemented as a variety of appropriate functions, for example, as follows:

$$E ( X ) = {\sum }_{i} {e}_{i} ( {x}_{i} ) + {\sum }_{i \neq j} {e}_{ij} ( {x}_{i} , {x}_{j} )$$

When the MRF model is suitable for data with appropriate regularization, the residue dependency in the virus protein sequence can be interpreted by the direct coupling term eij. Finally, the probability distribution of pairwise coupling can be calculated according to the following formula:

$${P}_{mut}^{msa} = softmax ( {W}^{msa} * {F}^{msa} )$$

$${F}_{i}^{msa} = [ {e}_{i} ( {x}_{i} ) ; {e}_{i 0} ( {x}_{i} , {x}_{0} ) , {e}_{i 1} ( {x}_{i} , {x}_{1} ) , … , {e}_{i n} ( {x}_{i} , {x}_{n} ) ]$$

wherein W^{msa} may represent trainable parameters. The above formula can be a functional representation of the mutated amino acid probability distribution predicted by multi-sequence alignment, which can correspond to the loss function in the prediction.

By way of example, training or determination may be implemented through a particular number of iterations. Then, when the result value of the loss function is less than a particular value, the iteration can terminate. Alternatively, the iteration can terminate after a specific number of iterations. Alternatively, in a case that the results of the loss function for a particular number of consecutive iterations is always below a particular value, or the difference between results of the loss function for a particular number of consecutive iterations is below a particular threshold, the iteration can terminate, a proper mutation condition can be set based on the loss function. Through this process, the prediction for amino acids, whether based on the background information of the virus protein sequence or the global coupling of MSA, is beneficial for better generation of virus variants.

Exemplary implementations of virus protein-related data processing according to embodiments of the present disclosure will be described in detail below with reference to the accompanying drawings, and in particular, the comparison and optimization of virus protein-related data processing according to embodiments of the present disclosure with respect to the prior art data processing will be described.

On one hand, the solution according to the present disclosure, EVPMM can accurately detect mutation position. In order to indicate the accuracy of EVPMM in the mutation position prediction, the proposed EVPMM is compared with six representative baselines, including statistical methods and neural network models.

The statistical method explicitly simulates the co-variation between all pairs of residues in the protein by fitting a statistical model for multi-sequence alignment (MSA) of all homologous sequences of the protein of interest: (1) MAFFT MSA uses an algorithm based on progressive alignment to create MSA for the amino acid sequences. (2) EVMutation is an unsupervised statistical model, which is also used as pairwise undirected graph models for multi-sequence alignment. (3) CSCS proposes a language model based on Istm to learn virus protein mutation semantics. (4) it further realizes a language model based on Transformer-Encoder as CSCS, and uses Masking Language Model (MLM) task to optimize it, which can be called as cross-marketing. (5) Trans-Mut: further realizing a transformer-encoder Trans-Mut with wild type as input and virus mutation type as output. (6) DeepSequence is a generation model for predicting mutation effect based on variation inference.

As can be seen from the above figures, given a fitted feature, EVPMM can better predict the advantageous amino acid at a particular position. As shown in FIG. 4B, EVPMM achieved the highest normalized AUC scores for all three virus data sets. This is because the amino acid prediction according to embodiments of the present disclosure is achieved by combining contextual virus protein information and global pairwise correlation from MSA, which facilitates better variant prediction. In most cases, the performance of the neural network model is superior to the statistical method, because deeper features captured by the neural network contribute to virus mutation prediction. The CSCS performs the worst in five neural network models, indicating that the model based on Transformer-Encoder is better than the model based on Istm in the aspect of virus variation prediction. Pairwise correlation facilitates virus mutation prediction. After removal of the pairwise dependency, scores of both AUC and r20 were significantly reduced, although the removal of the position predictor had a greater effect on performance. The result shows that the dependency relationship related to the residues in the virus protein sequence is beneficial for acquiring the high-order correlation, which can contribute to generation of rational virus variation.

On the other hand, EVPMM can accurately predict the functional fitness of the virus protein. The fitness refers to the relationship between the sequence and the function. When a new mutant is introduced, the function of the virus protein changes, resulting in increasing or decreasing of the fitness of the virus protein. Thus, accurate prediction of fitness is critical for early detection of variation and world health protection. In addition, the knowledge of the fitness of the virus protein plays a key role in the rational vaccine design.

As a comparison, EVPMM is compared with the DeepSequence models on the six HA data sets. As shown in FIG. 4C, EVPMM is superior to DeepSequence in performance over BK79, Bris07L194, Bei89, NDako16, Mos99 and all HA data sets. The result shows that EVPMM has strong adaptability to different virus variations. All these HA data sets are combinatorial mutation data sets, and the result shows that EVPMM has a stronger combinatorial mutation mode capturing ability than the existing SOTA model. As shown in FIG. 4C, EVPMM can accurately predict the fitness of the combined data set. The SARS-COV-2 data set contains a large number of single mutations and combinatorial mutations. On the single, double and all combinatorial mutation data sets, EVPMM has the same performance as DeepSequence. However, on the multi-site mutation data set with more than 3 mutation positions, EVPMM is superior to DeepSequence and other strong base lines. These results show that the EVPMM has strong fitting ability.

Some embodiments of the present disclosure further provide an electronic device that can be operable to implement the operations/functions of the above-mentioned model pre-training device and/or model training device. FIG. 5 is a block diagram of an electronic device according to some embodiments of the present disclosure. For example, in some embodiments, the electronic device 5 may be various types of devices, for example, may include, but not limited to, mobile terminals such as a mobile phone, a notebook computer, a digital broadcast receiver, a personal digital assistant (PDA), a tablet computer (PAD), a portable multimedia player (PMP), and a vehicle-mounted terminal (such as a vehicle navigation terminal), and fixed terminals such as a digital TV and a desktop computer. For example, the electronic device 5 may include a display panel configured to display data used in the solution according to the present disclosure and/or execution results. For example, the display panel may be in various shapes, such as a rectangular panel, an elliptical panel, or a polygonal panel, etc. In addition, the display panel may be a flat panel, a curved panel, or even a spherical panel.

As shown in FIG. 5, the electronic device 5 in this embodiment includes: a memory 51 and a processor 52 coupled to the memory 51. It should be noted that the components of the electronic device 50 shown in FIG. 5 are merely exemplary and non-limiting. The electronic device 50 may further have other components according to actual application requirements. The processor 52 may control other components in the electronic device 5 to perform desired functions.

In some embodiments, the memory 51 is configured to store one or more computer-readable instructions. The processor 52 is configured to run computer-readable instructions, and the computer-readable instructions, when run by the processor 52, cause the method according to any one of the above embodiments to be implemented. For specific implementations of the steps of the method and related explanation content, reference may be made to the above embodiments, and repetitions will not be repeated here.

For example, the processor 52 and the memory 51 may communicate with each other directly or indirectly. For example, the processor 52 and the memory 51 may communicate with each other via a network. The network may include a wireless network, a wired network, and/or any combination of wireless networks and wired networks. The processor 52 and the memory 51 may also communicate with each other through a system bus, which is not limited in the present disclosure.

For example, the processor 52 may be embodied as various appropriate processors, processing apparatuses, etc., such as a central processing unit (CPU), a graphics processing unit (GPU), and a network processor (NP); or may be a digital signal processor (DSP), an application-specific integrated circuit (ASIC), a field programmable gate array (FPGA) or other programmable logic devices, a discrete gate or transistor logic device, or a discrete hardware component. The central processing unit (CPU) may be of an X86 or ARM architecture, etc. For example, the memory 51 may include any combination of various forms of computer-readable storage media, for example, a volatile memory and/or a non-volatile memory. The memory 51 may include, for example, a system memory, and the system memory stores, for example, an operating system, an application, a boot loader, a database, and other programs. Various applications and various data may also be stored in the storage medium.

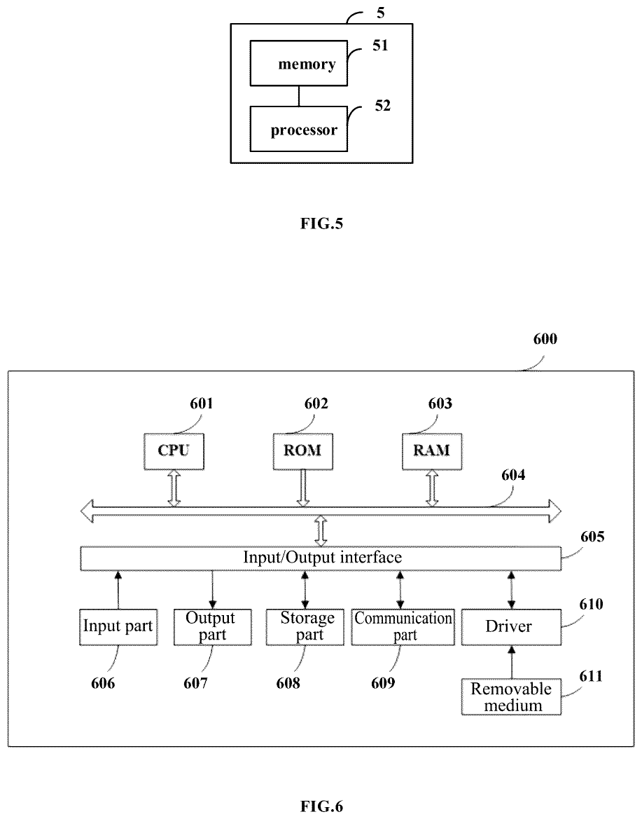

In addition, according to some embodiments of the present disclosure, when various operations/processing according to the present disclosure are implemented by software and/or firmware, programs constituting the software may be installed from the storage medium or a network to a computer system with a dedicated hardware structure, such as a computer system 600 shown in FIG. 6. When installed with various programs, the computer system can perform various functions, including the functions described above, etc. FIG. 6 is a block diagram of an example structure of a computer system that can be used according to an embodiment of the present disclosure.

In FIG. 6, a central processing unit (CPU) 601 performs various processing according to a program stored in a read-only memory (ROM) 602 or loaded from a storage part 608 into a random-access memory (RAM) 603. Data required for the CPU 601 to perform various processing and the like is also stored in the RAM 603 as required. The central processing unit is merely exemplary, and it may alternatively be other types of processors, such as the various processors described above. The ROM 602, the RAM 603, and the storage part 608 may be various forms of computer-readable storage media, as described below. It should be noted that although the ROM 602, the RAM 603, and the storage device 608 are shown separately in FIG. 6, one or more of them may be combined or located in the same or different memories or storage modules.

The CPU 601, the ROM 602, and the RAM 603 are connected to one another through a bus 604. An input/output interface 605 is also connected to the bus 604.

The following components are connected to the input/output interface 605: an input part 606, for example, a touch screen, a touchpad, a keyboard, a mouse, an image sensor, a microphone, an accelerometer, or a gyroscope; an output part 607, including a display, such as a cathode-ray tube (CRT), a liquid crystal display (LCD), a speaker, or a vibrator; the storage part 608, including a hard disk, a magnetic tape, etc.; and a communication part 609, including a network interface card, such as a LAN card, or a modem. The communication part 609 allows communication processing to be performed via a network such as the Internet. It is easy to understand that although the various apparatuses or modules in the electronic device 600 shown in FIG. 6 communicate through the bus 604, they may alternatively communicate through a network or other means, where the network may include a wireless network, a wired network, and/or any combination of wireless networks and wired networks.

A driver 610 is also connected to the input/output interface 605 as required. A removable medium 611 such as a magnetic disk, an optical disk, a magneto-optical disk, or a semiconductor memory, is installed on the driver 610 as required, such that a computer program read therefrom is installed into the storage part 608 as required.

When the above-described series of processing is implemented by software, programs constituting the software may be installed from a network such as the Internet or a storage medium such as the removable medium 611.

According to an embodiment of the present disclosure, the process described above with reference to the flowcharts may be implemented as a computer software program. For example, this embodiment of the present disclosure includes a computer program product, which includes a computer program carried on a computer-readable medium, where the computer program includes program code for performing the method according to the embodiments of the present disclosure. In such an embodiment, the computer program may be downloaded from a network through the communication device 609 and installed, installed from the storage device 608, or installed from the ROM 602. When the computer program is executed by the CPU 601, the above-mentioned functions defined in the method of the embodiments of the present disclosure are performed.

It should be noted that, in the context of the present disclosure, the computer-readable medium may be a tangible medium that may contain or store a program for use by or in conjunction with an instruction execution system, apparatus, or device. The computer-readable medium may be a computer-readable signal medium, a computer-readable storage medium, or any combination thereof. The computer-readable storage medium may be, for example, but is not limited to, an electric, magnetic, optical, electromagnetic, infrared, or semi-conductive system, apparatus, or device, or any combination thereof. A more specific example of the computer-readable storage medium may include, but is not limited to: an electrical connection having one or more wires, a portable computer magnetic disk, a hard disk, a random access memory (RAM), a read-only memory (ROM), an erasable programmable read-only memory (EPROM or flash memory), an optical fiber, a portable compact disk read-only memory (CD-ROM), an optical storage device, a magnetic storage device, or any suitable combination thereof. In the present disclosure, the computer-readable storage medium may be any tangible medium containing or storing a program which may be used by or in combination with an instruction execution system, apparatus, or device. In the present disclosure, the computer-readable signal medium may include a data signal propagated in a baseband or as a part of a carrier, the data signal carrying computer-readable program code. The propagated data signal may be in various forms, including but not limited to an electromagnetic signal, an optical signal, or any suitable combination thereof. The computer-readable signal medium may also be any computer-readable medium other than the computer-readable storage medium. The computer-readable signal medium can send, propagate, or transmit a program used by or in combination with an instruction execution system, apparatus, or device. The program code contained in the computer-readable medium may be transmitted by any suitable medium, including but not limited to: electric wires, optical cables, radio frequency (RF), etc., or any suitable combination thereof.

The above computer-readable medium may be contained in the above electronic device. Alternatively, the computer-readable medium may exist independently, without being assembled into the electronic device.

In some embodiments, there is further provided a computer program. The computer program includes instructions that, when executed by a processor, cause the processor to perform the method in any one of the above embodiments. For example, the instructions may be embodied as computer program code.

In the embodiments of the present disclosure, the computer program code for performing the operations of the present disclosure may be written in one or more programming languages or a combination thereof, where the programming languages include, but are not limited to, an object-oriented programming language, such as Java, Smalltalk, and C++, and further include conventional procedural programming languages, such as “C” language or similar programming languages. The program code may be completely executed on a computer of a user, partially executed on a computer of a user, executed as an independent software package, partially executed on a computer of a user and partially executed on a remote computer, or completely executed on a remote computer or server. In the case of the remote computer, the remote computer may be connected to the computer of the user via any kind of network, including a local area network (LAN) or a wide area network (WAN), or may be connected to an external computer (for example, connected via the Internet with the aid of an Internet service provider).

The flowcharts and block diagrams in the accompanying drawings illustrate the possibly implemented architecture, functions, and operations of the system, method, and computer program product according to various embodiments of the present disclosure. In this regard, each block in the flowchart or block diagram may represent a module, program segment, or part of code, and the module, program segment, or part of code contains one or more executable instructions for implementing the specified logical functions. It should also be noted that, in some alternative implementations, the functions marked in the blocks may also occur in an order different from that marked in the accompanying drawings. For example, two blocks shown in succession can actually be performed substantially in parallel, or they can sometimes be performed in the reverse order, depending on the functions involved. It should also be noted that each block in the block diagram and/or the flowchart, and a combination of the blocks in the block diagram and/or the flowchart may be implemented by a dedicated hardware-based system that executes specified functions or operations, or may be implemented by a combination of dedicated hardware and computer instructions.

The related modules, components, or units described in the embodiments of the present disclosure may be implemented by software, or may be implemented by hardware. The names of the modules, components, or units do not constitute a limitation on the modules, components, or units themselves in some cases.

The functions described herein above may be performed at least partially by one or more hardware logic components. For example, without limitation, exemplary hardware logic components that may be used include: a field programmable gate array (FPGA), an application-specific integrated circuit (ASIC), an application-specific standard product (ASSP), a system on a chip (SOC), a complex programmable logic device (CPLD), and the like.

The present disclosure may be implemented in any form described here, including but not limited to the example embodiments enumerated below, which describe structures, features, and functions of some parts of the embodiments of the present invention.

According to some embodiments of the present disclosure, there is provided a data processing method for virus protein mutation prediction, the method may include the following steps: acquiring relevant information about a virus protein sequence for prediction, wherein the relevant information includes amino acid type information, amino acid position information, and corresponding fitness information about the virus protein sequence, and based on the relevant information about the virus protein sequence for prediction, predicting an amino acid mutation site in the virus protein sequence by using a neural network.

In some embodiments, at least one of amino acid type information, amino acid position information, and corresponding fitness information in the virus protein sequence may be represented by an Embedding label manner.

In some embodiments, the fitness information may include a fitness tag sequence corresponding to the virus protein sequence, wherein each amino acid in the virus protein sequence has a corresponding fitness tag, and wherein the fitness tag can be a first specific value indicates that the mutation causes the fitness of the protein to become better, or a second specific value indicates that the mutation causes the fitness of the protein to become poor.

In some embodiments, predicting the amino acid mutation site in the virus protein sequence by using the neural network may include: encoding relevant information about the virus protein sequence with a particular number of encoder layers, wherein each encoder layer may include a self-attention block and a position-aware feed-forward block, and predicting the amino acid mutation site in the virus protein sequence based on information about the encoded virus protein sequence.

In some embodiments, predicting the amino acid mutation site in the virus protein sequence by using the neural network may include: predicting the amino acid mutation site in the virus protein sequence by using a mutation position prediction loss function based on SoftMax function.

In some embodiments, the method may further include: based on at least one of amino acid context information in the virus protein sequence and amino acid correlation information in the virus protein sequence, predict the amino acid mutation condition in the virus protein sequence at the predicted amino acid mutation site in the virus protein sequence by using the neural network.

In some embodiments, predicting the amino acid mutation condition in the virus protein sequence may include predicting probability distribution of an amino acid mutating to a particular amino acid type at the amino acid mutation site in the virus protein sequence by using a mutation prediction loss function based on SoftMax function.

In some embodiments, the mutation prediction loss function may be a weighted combination of a first loss function for prediction based on amino acid context information and a second loss function for prediction based on amino acid correlation information.

In some embodiments, the amino acid context information may include position information and amino acid relevant information of the predicted amino acid mutation site in the virus protein sequence, and the first loss function may be constructed based on the amino acid context information, a particular SoftMax function.

In some embodiments, residue dependency in the virus protein sequence can be obtained based on multiple sequence alignment for the virus protein sequence as the amino acid correlation information, and the second loss function may be constructed based on the residue dependency, a particular SoftMax function.

In some embodiments, residue dependency in the virus protein sequence can be obtained by: constructing an energy function for the virus protein sequence which can be constructed as a sum of all pairwise coupling constraints and single position constraints, and determining the residue dependency in the virus protein sequence based on at least the pairwise coupling constraint terms in the energy function.

In some embodiments, the amino acid context information and amino acid correlation information in a virus protein sequence may be acquired based on a homologous virus protein sequence, wherein the homologous virus protein sequence may be generated based on a virus protein sequence for predicting mutation sites in the virus protein sequence.

According to some embodiments of the present disclosure, there is provided a data processing apparatus for virus protein mutation prediction, comprising: an acquisition unit configured to acquire relevant information about a virus protein sequence for prediction, wherein the relevant information includes amino acid type information, amino acid position information, and corresponding fitness information about the virus protein sequence, and a mutation position prediction unit configured to, based on the relevant information about the virus protein sequence for prediction, predict an amino acid mutation site in the virus protein sequence by using a neural network.

In some embodiments, the data processing apparatus may further include: a mutation condition prediction unit configured to predict an amino acid mutation condition in the virus protein sequence at the predicted amino acid mutation site in the virus protein sequence by using the neural network, predict a mutation prediction loss function based on SoftMax function, wherein the amino acid mutation condition relates to a probability distribution of an amino acid mutating to a particular amino acid type.

According to some embodiments of the present disclosure, an electronic device is provided, which may include a memory; and a processor coupled to the memory, the memory having stored therein executable instructions that, when executed by the processor, cause the electronic device to perform the method according to any of the embodiments of the present disclosure.

According to still further embodiments of the present disclosure, there is provided a computer-readable storage medium having stored thereon a computer program that, when executed by a processor, implements the method described in any of the embodiments of the present disclosure.

According to yet another embodiment of the present disclosure, a computer program is provided, comprising: The instructions, when executed by the processor, cause the processor to perform the method described in any of the embodiments of the present disclosure.

According to some embodiments of the present disclosure, a computer program product is provided including instructions that, when executed by a processor, implement the method described in any of the embodiments of the present disclosure.

The foregoing descriptions are merely some embodiments of the present disclosure and explanations of the applied technical principles. Those skilled in the art should understand that the scope of disclosure involved in the present disclosure is not limited to the technical solutions formed by specific combinations of the foregoing technical features, and shall also cover other technical solutions formed by any combination of the foregoing technical features or equivalent features thereof without departing from the foregoing concept of disclosure. For example, a technical solution formed by a replacement of the foregoing features with technical features with similar functions disclosed in the present disclosure (but not limited thereto) also falls within the scope of the present disclosure.

In the description provided herein, numerous specific details are set forth. However, it is understood that the embodiments of the present invention may be practiced without these specific details. In other cases, well-known methods, structures, and techniques have not been shown in detail in order not to obscure the understanding of this description.

In addition, although the various operations are depicted in a specific order, it should be understood as requiring these operations to be performed in the specific order shown or in a sequential order. Under certain circumstances, multitasking and parallel processing may be advantageous. Similarly, although several specific implementation details are included in the foregoing discussions, these details should not be construed as limiting the scope of the present disclosure. Some features that are described in the context of separate embodiments can also be implemented in combination in a single embodiment. In contrast, various features described in the context of a single embodiment may alternatively be implemented in a plurality of embodiments individually or in any suitable sub combination.

While some specific embodiments of the present disclosure have been exemplarily described in detail, it should be understood by those skilled in the art that the above examples are merely for illustration and are not intended to limit the scope of the present disclosure. Those skilled in the art should understand that various modifications can be made to the above embodiments, without departing from the scope and spirit of the present disclosure. The scope of the present disclosure is defined by the appended claims.

What is claimed is:

1. A data processing method for virus protein mutation prediction, comprising:
acquiring relevant information about a virus protein sequence for prediction, wherein the relevant information includes amino acid type information, amino acid position information, and corresponding fitness information in the virus protein sequence, and
based on the relevant information in the virus protein sequence for prediction, predicting an amino acid mutation site in the virus protein sequence by using a neural network.

2. The data processing method of claim 1, wherein, at least one of amino acid type information, amino acid position information, and corresponding fitness information in the virus protein sequence is represented by an Embedding label manner.

3. The data processing method of claim 2, wherein, the fitness information comprises a fitness tag sequence corresponding to the virus protein sequence, wherein each amino acid in the virus protein sequence has a corresponding fitness tag, and wherein the fitness tag can be a first specific value indicates that the mutation causes the fitness of the protein to become better, or a second specific value indicates that the mutation causes the fitness of the protein to become poor.

4. The data processing method of claim 1, wherein, the predicting the amino acid mutation site in the virus protein sequence by using the neural network comprises:
encoding relevant information about the virus protein sequence with a particular number of encoder layers, wherein each encoder layer comprises a self-attention block and a position-aware feed-forward block, and
predicting the amino acid mutation site in the virus protein sequence based on information about the encoded virus protein sequence, and/or
wherein, the predicting the amino acid mutation site in the virus protein sequence by using the neural network comprises:
predicting the amino acid mutation site in the virus protein sequence by using a mutation position prediction loss function based on SoftMax function.

5. The data processing method of claim 1, further comprising:
based on at least one of amino acid context information in the virus protein sequence and amino acid correlation information in the virus protein sequence, predicting an amino acid mutation condition in the virus protein sequence at the predicted amino acid mutation site in the virus protein sequence by using the neural network.

6. The data processing method of claim 5, wherein, the predicting the amino acid mutation condition in the virus protein sequence comprises:
predicting probability distribution of an amino acid mutating to a particular amino acid type at the amino acid mutation site in the virus protein sequence, by using a mutation prediction loss function based on SoftMax function.

7. The data processing method of claim 6, wherein, the mutation prediction loss function is a weighted combination of a first loss function for prediction based on amino acid context information and a second loss function for prediction based on amino acid correlation information.

8. The data processing method of claim 7, wherein, the amino acid context information comprises position information and amino acid relevant information of the predicted amino acid mutation site in the virus protein sequence, and the first loss function is constructed based on the amino acid context information, a particular SoftMax function, and/or
wherein, residue dependency in the virus protein sequence is obtained based on multiple sequence alignment for the virus protein sequence as the amino acid correlation information, and the second loss function is constructed based on the residue dependency, a particular SoftMax function.

9. The data processing method of claim 8, wherein, the obtaining the residue dependency in the virus protein sequence comprises:
constructing an energy function for the virus protein sequence which is constructed as a sum of all pairwise coupling constraints and single position constraints, and
determining the residue dependency in the virus protein sequence based on at least the pairwise coupling constraint terms in the energy function.

10. The data processing method of claim 5, wherein, the amino acid context information and amino acid correlation information in a virus protein sequence is acquired based on a homologous virus protein sequence, wherein the homologous virus protein sequence may be generated based on a virus protein sequence for predicting mutation sites in the virus protein sequence.

11. An electronic device, comprising: a memory; and a processor coupled to the memory, the memory storing executable instructions, which, when executed by the processor, cause the processor to implement:
acquiring relevant information about a virus protein sequence for prediction, wherein the relevant information includes amino acid type information, amino acid position information, and corresponding fitness information in the virus protein sequence, and
based on the relevant information in the virus protein sequence for prediction, predicting an amino acid mutation site in the virus protein sequence by using a neural network.

12. The electronic device of claim 11, wherein, the predicting the amino acid mutation site in the virus protein sequence by using the neural network comprises:
encoding relevant information about the virus protein sequence with a particular number of encoder layers, wherein each encoder layer comprises a self-attention block and a position-aware feed-forward block, and
predicting the amino acid mutation site in the virus protein sequence based on information about the encoded virus protein sequence, and/or
wherein, the predicting the amino acid mutation site in the virus protein sequence by using the neural network comprises:
predicting the amino acid mutation site in the virus protein sequence by using a mutation position prediction loss function based on SoftMax function.

13. The electronic device of claim 11, wherein, the executable instructions, when executed by the processor, cause the processor to further implement:
based on at least one of amino acid context information in the virus protein sequence and amino acid correlation information in the virus protein sequence, predicting an amino acid mutation condition in the virus protein sequence at the predicted amino acid mutation site in the virus protein sequence by using the neural network.

14. The electronic device of claim 13, wherein, the predicting the amino acid mutation condition in the virus protein sequence comprises:
predicting probability distribution of an amino acid mutating to a particular amino acid type at the amino acid mutation site in the virus protein sequence, by using a mutation prediction loss function based on SoftMax function.

15. The electronic device of claim 14, wherein, the mutation prediction loss function is a weighted combination of a first loss function for prediction based on amino acid context information and a second loss function for prediction based on amino acid correlation information,
wherein, the amino acid context information comprises position information and amino acid relevant information of the predicted amino acid mutation site in the virus protein sequence, and the first loss function is constructed based on the amino acid context information, a particular SoftMax function, and/or
wherein, residue dependency in the virus protein sequence is obtained based on multiple sequence alignment for the virus protein sequence as the amino acid correlation information, and the second loss function is constructed based on the residue dependency, a particular SoftMax function.

16. A non-transitory computer-readable storage medium having stored thereon executable instructions which, when executed by a processor, cause the processor to implement:
acquiring relevant information about a virus protein sequence for prediction, wherein the relevant information includes amino acid type information, amino acid position information, and corresponding fitness information in the virus protein sequence, and
based on the relevant information in the virus protein sequence for prediction, predicting an amino acid mutation site in the virus protein sequence by using a neural network.

17. The non-transitory computer-readable storage medium of claim 16, wherein, the predicting the amino acid mutation site in the virus protein sequence by using the neural network comprises:
encoding relevant information about the virus protein sequence with a particular number of encoder layers, wherein each encoder layer comprises a self-attention block and a position-aware feed-forward block, and
predicting the amino acid mutation site in the virus protein sequence based on information about the encoded virus protein sequence, and/or
wherein, the predicting the amino acid mutation site in the virus protein sequence by using the neural network comprises:
predicting the amino acid mutation site in the virus protein sequence by using a mutation position prediction loss function based on SoftMax function.

18. The non-transitory computer-readable storage medium of claim 16, wherein, the instructions, when executed by the processor, cause the processor to further implement:
based on at least one of amino acid context information in the virus protein sequence and amino acid correlation information in the virus protein sequence, predicting an amino acid mutation condition in the virus protein sequence at the predicted amino acid mutation site in the virus protein sequence by using the neural network.

19. The non-transitory computer-readable storage medium of claim 18, wherein, the predicting the amino acid mutation condition in the virus protein sequence comprises:
predicting probability distribution of an amino acid mutating to a particular amino acid type at the amino acid mutation site in the virus protein sequence, by using a mutation prediction loss function based on SoftMax function.

20. The non-transitory computer-readable storage medium of claim 19, wherein, the mutation prediction loss function is a weighted combination of a first loss function for prediction based on amino acid context information and a second loss function for prediction based on amino acid correlation information,
wherein, the amino acid context information comprises position information and amino acid relevant information of the predicted amino acid mutation site in the virus protein sequence, and the first loss function is constructed based on the amino acid context information, a particular SoftMax function, and/or
wherein, residue dependency in the virus protein sequence is obtained based on multiple sequence alignment for the virus protein sequence as the amino acid correlation information, and the second loss function is constructed based on the residue dependency, a particular SoftMax function.
